(19) United States
(12) Patent Application Publication
(10) Pub. No.: US 2005/0009862 A1
(43) Pub. Date: Jan. 13, 2005

(54) Method for promoting uninterrupted sleep by administration of trospium chloride

(76) Inventors: LuAnn Sabounjian, Holliston, MA (US); Bobby W. Sandage JR., Acton, MA (US)

Correspondence Address: PATENT ADMINSTRATOR;KATTEN MUCHIN ZAVIS ROSENMAN, 525 WEST MONROE STREET, SUITE 1600, CHICAGO, IL, 60661-3693, US

(21) Appl. No.: 10/831,131

(22) Filed: Apr. 26, 2004

Related U.S. Application Data
(60) Provisional application No. 60/465,208, filed on Apr. 25, 2003.

(51) Int. Cl. A61K 31/46

(52) U.S. Cl. 514/304

(57) ABSTRACT

A method of promoting sleep in a patient with overactive bladder, comprising: administering trospium chloride at or shortly before bedtime, to a patient with overactive bladder disease, in an amount sufficient to reduce wakefulness throughout the patient's normal sleep period.

### FIELD OF THE INVENTION

The present invention relates to a method for promoting uninterrupted sleep by administration of trospium chloride. In particular, the invention facilitates sleep and rest by allowing a person to sleep through the night without having to awaken, get up, and urinate in a toilet. The present method is particularly useful for persons who find that they have to interrupt their sleep and go to the bathroom several times during the night.

### BACKGROUND OF THE INVENTION

Trospium chloride is an agent that has been known for many years (cf. German patent 1 194 422) as an anticholinergic that is useful as a spasmolytic agent. The active agent has been available as an orally administrable solid form as tablets, for intravenous or intramuscular injection as a solution (Schwantes et al., U.S. Pat. No. 5,998,430) and for rectal administration as suppositories. It is mainly used for the treatment of bladder dysfunctions.

Trospium chloride was known to reduce urinary frequency in patients with overactive bladder. It has now been discovered that trospium chloride reduces the severe feeling of urgency or discomfort that often accompanies any need to urinate. Reduced discomfort also unexpectedly alleviates a tendency in these patients to awaken repeatedly during the night.

Urgency severity can be evaluated for different drugs using the Indevus Urgency Severity Scale (IUSS) developed by Indevus Pharmaceuticals, Inc., described below.

Identification of a therapy that greatly reduces feelings of urgency to urinate while sleeping provides a way to treat a problem for the subpopulation of overactive bladder patients who also experience disrupted sleep patterns even though their condition is being treated, which causes them to wake up repeatedly throughout the night with a strong urge to urinate.

Although trospium chloride has been used to treat bladder dysfunction, the unexpected effect of trospium chloride in promoting uninterrupted sleep by preventing episodes of wakefulness at night has not been previously used. The present invention is directed to methods which rely upon this surprising effect to reduce or eliminate sleep disruptions in normal people as well as people with overactive bladder disease.

### SUMMARY OF THE INVENTION

The present invention provides a method for promoting uninterrupted sleep in general, and in particular in patients with overactive bladder disease. The method comprises administering trospium chloride or another trospium salt before bedtime in an amount sufficient to inhibit awakening during the person's normal sleep period.

The present invention also provides a method for measuring the reduction in severity of the urge to urinate caused by a test compound administered to patients suffering from overactive bladder disease. This effect is distinct from the frequency of urination and also distinct from simple feeling of a need to urinate. In some patients with overactive bladder disease, any need to urinate at all is perceived as an overwhelming sensation that interrupts all other activities. The present invention provides a method for evaluating therapies for this particular form of the condition.

### DETAILED DESCRIPTION OF THE INVENTION

In the following description, reference will be made to various methods well known to those skilled in the art of medicine and pharmacology. Such methods arc described in standard reference works setting forth the general principles of these disciplines.

One can inhibit a person from awakening during the night by administering an effective amount of trospium shortly before sleep, specifically 0.5 h, 1.0 h, 1.5 h or 2.0 h before bedtime. This method is particularly suited to people with overactive bladder, but can also be used by people in general to promote uninterrupted sleep.

The invention also provides a method for reducing the severity of the urge of a person suffering from overactive bladder to urinate. This effect is distinct from reducing the frequency of urination episodes. We have found the compound reduces feelings of urgency to an unexpected degree.

Any route of delivery may be used for administering the trospium chloride before sleep to a patient with overactive bladder disease. The treatment reduces wakefulness and provides uninterrupted sleep. Preferred routes include oral administration by tablets or capsules. Administration transdermally, intravenously, buccally, or sublingually are also possible. The preferred oral dose is between 1 mg/kg per day and 75 mg/kg per day, given at or just before bedtime. When the oral route of delivery is used, the preferred dosage range is between 10 mg/kg and 60 mg/kg, typically between about 20 and 50 mg per day. The dosage for transdermal, buccal and sublingual routes of administration should be between 1.0 mg/kg per day and 75 mg/kg per day.

These dosage ranges are simply guidelines since the actual dose may be selected and titrated by the attending physician based upon clinical conditions. The optimal daily dose will be determined by methods known in the art and will be influenced by factors such as the age of the patient, the condition or disease associated with the overactive bladder condition, the severity of both the urge to urinate and the condition of the patient to whom treatment is being given, the desired degree of therapeutic response, and the concomitant therapies being administered. Dosages may be provided in either a single or multiple dosage regimen, although one dose shortly before sleep is preferred.

Trospium is also known by the specific chemical name 3-alpha-benziloyloxynortropane-8-spiro-1′-pyrrolidinium chloride, or any other trospium salt. The present invention also provides a trospium chloride composition adapted for administration just before bedtime in a single dose.

Dosage Forms and Route of Administration

Any route of administration and dosage form is compatible with the present invention. There are numerous references that provide guidance in this respect; for example, U.S. Pat. No. 4,812,481 discloses therapeutic dosage forms in which an active ingredient is administered in oral, peroral, internal, pulmonary, rectal, nasal, vaginal, lingual, intravenous, intraarterial, intracardial, intramuscular, intraperitoneal, intracutaneous, and subcutaneous formulations. U.S. Pat. No. 5,192,550 describes a dosage form for an active ingredient comprising an outer wall with one or more pores, in which the wall is impermeable to the drug but permeable to external fluids. This dosage form may have applicability for oral, sublingual, or buccal administration. Similarly, U.S. Pat. No. 5,387,615 discloses a variety of pharmaceutical compositions, including tablets, pills, capsules, powders, aerosols, suppositories, skin patches, parenterals, and oral liquids, including oil aqueous suspensions, solutions, and emulsions. Also disclosed therein are sustained release (long acting) formulations and devices. All of the U.S. patents identified above are incorporated herein by reference in their entirety.

Parenteral compositions containing trospium chloride may be prepared according to conventional techniques. For example, sterile isotonic saline may be used in preparations designed for intramuscular, intravenous, intrathecal or intraarterial delivery.

Transdermal dosage unit forms can be prepared using a variety of techniques that have been described. For example, in U.S. Pat. Nos. 4,861,800; 4,868,218; 5,128,145; 5,190,763; and 5,242,950 (each incorporated herein by reference in their entirety); and in the foreign patent documents EP-A 404807; EP-A 509761; and EP-A 593807. A monolithic patch structure can be utilized in which trospium chloride is directly incorporated into the adhesive and this mixture is cast on a backing sheet. One can also employ a device using a lyotropic liquid crystalline composition in which, for example, 5-15% of trospium chloride is combined with a mixture of liquid and sold polyethylene glycols, a polymer, and a non-ionic surfactant, optionally with the addition of propylene glycol and an emulsifying agent. For further details on the preparation of such transdermal formulations, reference can be made to EP-A 5509761.

Buccal and sublingual dosage forms of trospium chloride may be prepared utilizing techniques similar to those described in U.S. Pat. Nos. 5,192,550; 5,221,536; 5,266,332; 5,057,321; 5,446,070; 4,826,875; 5,304,379; or 5,354,885.

Form of Trospium

The present invention is not limited to a particular form of trospium and the drug may be used in the form of other salts, including bromide, phosphate and sulfate.

The examples below are for illustrative purposes only and are not intended to limit the scope of the invention.

### EXAMPLE 1

Introduction

Overactive bladder is a medical condition characterized by the symptoms of urinary urgency and urge incontinence which are often associated with urinary frequency and nocturia that appear without a local pathologic or metabolic explanationⁱ.
ⁱAbrams P, Cardozo L, Fall M, et al. The standardization of terminology of lower urinary tract function: Report from the standardization sub-committee of the International Continence Society. Neurourol Urodyn 2002; 21: 167-178.

The Indevus (single-item) Urgency Scale requires a ‘quality assessment’ to ensure that it is a valid, reliable and responsive patient-reported outcome assessment tool. Psychometric methods used to evaluate new and adapted patient-reported outcome instruments include standard procedures for the assessment of reliability, validity and responsiveness. Psychometric assessment is not only an important stage in the development or adaptation of patient-reported outcome instruments, but the process will also bolster the credibility of results obtained in future studies.

Methods

2.1 Study Data

A multi-center, parallel, randomized, double-blind, placebo controlled study (#IP631-003) was based on data collected as a result of administering the IUSS during a 12-week¹-clinical trial of patients with overactive bladder (OAB) associated with predominant urge incontinence—for trospium chloride in the United States. For full details on the clinical study ethics, administration, objectives and design, selection of study population, treatments, efficacy and safety variables and statistical methodology please refer to the clinical study protocol #IP631-003 and the ‘Final Report for the Double-Blind Phase’ prepared by Quintiles Inc.

2.2 Indevus Urgency Severity Scale

The IUSS (Appendix A) asks patients about their ‘degree of urgency’ of their toilet voids (as meant to describe the urge to urinate). Patients are asked to rate the degree of urgency they felt before reaching the toilet for a toilet void (defined in the study as urination in the toilet). The instructions state that “Sometimes you may feel a very strong urge to urinate, and at other times, you may feel a milder urge prior to the onset of a toilet void. Rate this feeling by circling 0, 1, 2 or 3.” The instructions specify the following four response options:
0: None—no urgency;
1: Mild—awareness of urgency, but is easily tolerated and you can continue with your usual activity or tasks;
2: Moderate—enough urgency discomfort that it interferes with or shortens your usual activity or tasks; and
3: Severe—extreme urgency discomfort that abruptly stops all activity or tasks.

This single-item Urgency Severity Scale was contained within a ‘Patient Urinary Diary’, which was completed by all patients during four discrete one-week periods in the course of the 12-week double-blind trial (baseline and study treatment weeks 1, 4 and 12). During each of the one-week data collection periods, patients completed the diary every day for a total of seven days. The diary also collected data on the number of toilet voids and whether the patient had an accidental urge or stress leak. In the diary, an urge leak was defined as a leakage due to a very strong need or desire to urinate. A stress leak was defined as being a result of coughing, sneezing, standing, laughing, exercise, or other physical activity/movement exerting pressure on the bladder. In addition, during the last two days of the 7-day diary collection period (days 6 and 7), patients collected and measured all urine volume for each and every toilet void and recorded this within the diary.

2.3 Study Population

The study population for the clinical trial was 523 patients who received at least one dose of the study medication. Patients who entered the trial were required to have overactive bladder, defined as:
Urinary frequency of ≧70 toilet voids per 7 days (i.e. ≧10 toilet voids per day) as recorded in a patient urinary diary during a “washout” period;
Symptoms of urgency (i.e. sudden desire to toilet void);
Pure urge or mixed urinary incontinence with predominant urge incontinence. Patients must have had a minimum of 7 urge incontinence episodes per 7 days (i.e. average ≧1 urge incontinence episode per day).

It was a requirement that patients had experienced the symptoms of overactive bladder for a minimum of six months. It was also a requirement that patients were aged ≧18 years. Patients were excluded if they had stress incontinence, insensate incontinence and/or overflow incontinence as the major reason for urine loss of urinary frequency. Patients were also excluded if they had a history of neurogenic bladder, significant renal disease, uninvestigated hematuria and/or patients with acute urinary tract infections. For the exact inclusion and exclusion criteria please refer to study protocol #IP631-003. For the clinical study, subjects were randomized to one of two treatment groups, however for the purpose of the psychometric analysis all subjects were pooled into one cohort. In addition, sensitivity was conducted to ensure that the inclusion of the trospium group into the patient cohort at week 12 was a valid approach by running the analysis on the trospium patient group and comparing the p-values from this analysis with the results from analysis for the entire patient cohort (trospium chloride and placebo).

2.4 Study Medication

Trospium chloride is an anticholinergic agent with predominantly peripheral antimuscarinic activity. Its mechanism enables the detrusor to relax, inhibiting evacuation of the bladder. Patients were randomized on a 1:1 basis to receive either placebo or trospium chloride. Patients receiving trospium chloride took one 20-mg tablet twice daily. Those receiving placebo took one placebo tablet twice daily. The identity of the study medication was blinded.

2.5 Patient-Reported Outcome Data

Patients completed a patient diary on a daily basis during four one-week data collection windows: baseline, week 1, week 4 and week 12. The diary collected the following patient-reported outcome data:
IUSS;
Number of toilet voids;
Number of urge incontinence episodes, and separately, stress incontinence episodes;
Volume voided (mL), collected on the last 2 of 7 days of the 7 day diary.

In addition, patients completed either the female (standard IIQ) or male (modified IIQ) versions of the Incontinence Impact Questionnaireⁱⁱ (IIQ) at baseline and at week 12. The IIQ was used to assess the impact of OAB and urge incontinence and the effects of treatment on the patient's quality of life. For more details on the outcome data listed above please refer to protocol #IP631-003 and the Integrated Clinical and Statistical Report prepared by Quintiles, Inc.
ⁱⁱShumaker S. A., Wyman J. F., Vebersax J. S., McClish D, Fanti J. A., for the Continence Program in Women (CPW) Research Group. Health-related quality of life measures for women with urinary incontinence: the Incontinence Impact Questionnaire and the Urogenital Distress Inventory. Quality of Life Research 1994; 3: 291-306.

The data collected during the baseline and week 12 data collection periods was used to assess the psychometric properties of the IUSS.

2.6 Data Management

The raw and analytic data sets containing the results from the #IP631-003 clinical study was provided by Quintiles, Inc., Research Triangle Park on behalf of Indevus Pharmaceuticals. The data was provided in SAS format. The observed cases (OC) data set was used to conduct the psychometric analysis rather than the last observation carried forward (LOCF) data set. Quintiles Late Phase used SAS® Version 8.2 to analyze the data for the validation of the IUSS.

2.7 Levels of significance

The p-values will be based on two-tailed tests of significance. P-values ≦0.05 will be considered significant; p-values >0.05 and ≦0.10 will be thought to represent a trend towards significance.

2.8 Data Quality Assurance and Quality Control

The psychometric validation of the IUSS was based on data collected during the clinical trial protocol #IP631-003. The standardization procedure, data quality assurance and monitoring and auditing procedures are detailed in the Integrated Clinical and Statistical Report.

The quality control and assessment of the Psychometric Evaluation of an Urgency Severity Scale in Patients with Overactive Bladder: A Retrospective Validation (#IP631-008) study was ensured through standard quality control procedures followed by Quintiles, Inc. In summary, the feasibility assessment and statistical analysis plan (#IP631-008 Version 3, Mar. 6, 2003) underwent internal review, and the SAS programming and results were quality checked by a senior biostatistician.

2.9 Calculating Patient-Reported Outcome Scores

2.9.1 Urgency Scale

The IUSS (Appendix A) was completed on a daily basis for four one-week data collection periods. The Urgency Scale variable was utilized from the clinical trial study database (#IP631-003), where the average of all entries over baseline and week 12 was calculated. The data was set to missing if entries were not made for at least 4 full days of the 7 required days. If only 4, 5 or 6 days of entries were available, they were normalized to 7 days.

2.9.2 Toilet Voids

The number of toilet voids was completed on a daily basis for four one-week data collection periods. The Toilet Voids variable was utilized from the clinical trial study database (#IP631-003), where the average number of entries over baseline and week 12 was computed. The data was set to missing if entries were not made for at least 4 full days of the 7 required days. If only 4, 5 or 6 days of entries were available, they were normalized to 7 days.

2.9.3 Urge Incontinence Episodes

The number of urge incontinence episodes was completed on a daily basis during the four one-week data collection periods. The Urge Incontinence variable was utilized from the clinical trial study database (#IP631-003), where the average number of entries over baseline and week 12 was computed. The data was set to missing if entries were not made for at least 4 full days of the 7 required days. If only 4, 5 or 6 days of entries were available, they were normalized to 7 days.

2.9.4 Volume Voided

The volume voided (mL) was collected for 2 full days prior to each study visit (day 6 and day 7 of the patient urinary diary collection). The Volume Voided variable was utilized from the clinical trial study database (#IP631-003), where the average volume voided per toilet void for a patient for baseline and week 12 was calculated. This was only calculated if entries were made for both of the required days. If this information was not available, this patient's data was set to missing.

2.9.5 Incontinence Impact Questionnaire

The IIQ (Appendix B and C) was scored as detailed in the Integrated Clinical and Statistical Report for the #IP631-003 study. The four sub-scale scores were obtained by taking the mean value of all items responded to within each sub-scale. These scores were then to be transformed by subtracting 1 and multiplying times 100/3 to put them onto a common scale of 0 to 100. The total IIQ score was calculated by summing the four sub-scale scores, with a total possible of 0 to 400. This scoring system gave equal weight to each sub-scale. Analysis was undertaken on the gender-specific questionnaires and on combined data sets for each IIQ sub-scale score (physical activity, travel, social relationships, emotional health) and for IIQ total score. Gender specific analysis was undertaken because the male version of the IIQ had not been fully validated and it had not been confirmed that it is valid to combine results from the male and female versions.

2.9.6 Construct validity

There were four criterion measures for assessing the construct validity of the IUSS:
Urinary frequency—the average number of toilet voids per day for the 7 day period;
Urge Incontinence Episodes—the average number of urge incontinence episodes per day for the 7 day period;
Volume—the average volume voided (in mL) per toilet void, collected for 2 days
Quality of life—total score and subscale scores (physical activity, travel, social relationships, emotional health) on the Incontinence Impact Questionnaire (male, female and combined).
   Scores from the criterion instruments were correlated with the Indevus Urgency Severity Scale as listed above using Spearman's rho and Pearson's correlation coefficients. This correlation was assessed for the baseline and week 12 treatment periods.
   2.9.7 Known-Groups Validity

Known group validity was assessed twice, using two efficacy variables to determine the known groups:
1. Average number of toilet voids per day; and
2. Average number of urge incontinence episodes.
   The cut-off point for identifying the known groups was the median score for each of the two efficacy variables listed above. Patients who score the median score were placed into the higher group. This was assessed for the baseline and week 12 data. The respondent's IUSS score for the two groups for both efficacy variables was compared using the Mann-Whitney U test and the CMH test (with pooled center as the stratification variable).
   2.9.8 Responsiveness

Responsiveness of the IUSS was evaluated utilizing the effect size calculation. Responsiveness was assessed three times using three outcomes as external criteria to classify the patients as responders or not.
1. Normal Void Frequency Outcome:
   Patients were classified as responders if they had an average of ≦7 toilet voids per 24 hours during week 12.
   Patients were classified as non-responders if they had an average of >7 toilet voids per 24 hours during week 12.
2. Urge Incontinence Responder Outcome:
   Patients were classified as responders if they had an average of <1 urge incontinence event per 24 hours during week 12.
   Patients were classified as non-responders if they had ≦1 urge incontinence event per 24 hours during week 12.
3. Complete Responder Outcome;
   Patients were classified as responders if they had an average of ≦7 toilet voids and an average of <1 urge incontinence event per 24 hours during week 12.
   Patients were classified as non-responders if they had an average of >7 toilet voids and/or an average of ≦1 urge incontinence events per 24 hours during week 12 (i.e. could meet either or both criteria).
      An effect size of 0.20 was considered small, 0.50 moderate and 0.80 largeⁱⁱⁱ.
      ⁱⁱⁱCohen J. Statistical power analysis for the behavioral sciences. New York: Academic Press, 1977:8.
      2.9.9 Test-Retest Reliability

Correlating the average daily score on the IUSS for each patient on baseline day 1 and baseline day 7 was used to assess the test-retest reliability. Using the baseline data ensured that responses were not influenced by side effects, efficacy, study therapy, and compliance, which would have been an issue if data between weeks 1 and 12 were used. Scores were compared using Spearman's rho and Pearson's correlation coefficients.

Results

Descriptive statistics for each outcome utilized during the psychometric evaluation of the IUSS are presented in Table 1 (Appendix D). In summary analysis was conducted on 523 patients at baseline (523 patients were randomized and dispensed study medication. 520 patients received at least one dose of study medication.), the mean score on the IUSS was 1.77 (SE 0.54), with scores ranging across the entirety of the scale (from 0 to 3). The average number of toilet voids per 24 hours was 12.85 (SE 2.60), with a minimum of 9.29 and a maximum of 23.14. The average number of urge incontinence episodes per 24 hours was 4.11 (SE 3.13), with a minimum of 0.86 and a maximum of 21.29. The average volume voided per toilet void was 155.27 ml (SE 49.29), with a minimum of 20.18 and a maximum of 286.11. The mean IIQ total score was 190.56 (SE 3.77) ranging from 0 to 388.89. The mean IIQ total score for females was slightly higher (e.g. worse quality of life) (mean 197.13, (SE 4.36) compared to males (mean 171.58, SE 7.29). Female patients also scored higher (worse quality of life) on each of the IIQ subscales compared to male patients.

Analysis was conducted on all patients with available (observed cases) data at week 12. Using the IUSS, patients had a mean average urgency severity associated with toilet voids rating of 1.62 (SE 0.65), ranging from 0 to 3. The average number of toilet voids per 24 hours was 10.80 (SE 2.95), with a minimum of 4.29 and a maximum of 24.86. The average number of urge incontinence episodes per 24 hours was 1.83 (SE 2.56), with a minimum of 0 and a maximum of 14.57. The average volume voided per toilet void was higher than during the baseline period (178.32 ml, SE 67.16), with a minimum of 29.58 and a maximum of 404.09. The mean IIQ total score was 143.02 (SE 4.53) ranging from 0 to 400. The mean IIQ total score for females was slightly higher (e.g. worse quality of life) (mean 146.59, SE 5.37) compared to males (mean 132.56, SE 8.24). As for the baseline data, female patients also scored higher (worse quality of life) on each of the IIQ subscales compared to male patients.

3.1 Construct Validity

The results from assessing the construct validity of the IUSS are presented in Tables 2a (Pearson's correlation coefficient) and 2b (Spearman's rho) (Appendix D).

At baseline, the IUSS had a significant positive correlation (p≦0.05), with 12 of the 18 outcomes when this was assessed using the Pearson Product Moment [and Spearman's rho] correlation coefficients:
Average number of toilet voids per 24 hours;
Average Number of Urge Incontinence Episodes per 24 hours;
IIQ Total Score (Female and Combined);
IIQ Physical Activity subscale score (Female and Combined);
IIQ Travel subscale score (Female and Combined);
IIQ Social Relationships subscale score (Female and Combined);
IIQ Emotional Health subscale score (Females and Combined).

The correlation coefficients ranged from 0.18 (IIQ Physical Activity subscale score−Female) to 0.34 (Average Number of Urge Incontinence Episodes per 24 hours) when the Pearson Product Moment correlation coefficient was applied. For the 12 outcomes above, correlation coefficients were the same (n=7) or similar (n=5) when this was assessed using Spearman's rho. Correlation coefficients were not significant but the p-values suggested a trend towards significance with 3 outcomes when this was assessed using the Pearson Product Moment [and Spearman's rho] correlation coefficients:
IIQ Total Score (Male);
IIQ Physical Activity subscale score (Male);
IIQ Emotional Health subscale score (Male).
   Three of the outcomes were not significant with no suggestion of a trend towards significance:
Average Volume Voided (in mL) per toilet void;
IIQ Travel subscale score (Male);
IIQ Social Relationships subscale score (Male);

At week 12, the IUSS had a significant positive correlation (p≦0.05) with 17 of the 18 outcomes when correlation was assessed using the. Pearson Product Moment [and Spearman's rho] correlation coefficients:
Average Number of Toilet Voids per 24 hours;
Average Number of Urge Incontinence Episodes per 24 hours;
IIQ Total score (Female, Male and Combined);
IIQ Physical Activity subscale score (Female, and Combined);
IIQ Travel subscale score (Female, Male and Combined);
IIQ Social Relationships subscale score (Female, male and Combined);
IIQ Emotional Health subscale score (Female, male and Combined).
   The correlation coefficients ranged from 0.20 (IIQ Travel subscale score−Male) to 0.34 (Average Number of Urge Incontinence Episodes per 24 hours) when the Pearson Product Moment correlation coefficient was applied. For the 17 outcomes above, correlation coefficients were the same (n=3) or similar (n=14) when correlation was assessed using Spearman's rho. Only the average volume voided (in mL) per toilet void did not have a significant relationship with the IUSS when applying either the Pearson Product Moment or Spearman's rho correlation coefficients.

3.2 Known-Groups Validity

The results from assessing the known-groups validity of the IUSS are presented in Table 3 (Appendix D). These results show that the IUSS discriminated between patients who had above and below the median number of toilet voids per 24 hours during baseline and week 12 (p<0.01). The IVSS also distinguished between patients who had above and below the median number of urge incontinence episodes per 24 hours for the baseline and week 12 periods (p<0.01). The CMH test result revealed that patients who were categorized as Group A at baseline remained in Group A at week 12, and likewise for the Group B patients, for both the known group assessments.

3.3 Responsiveness

Responsiveness of the IUSS was evaluated using the effect size calculation, which transforms the score change into a standard unit of measurement to enable it to be compared with score changes on other instruments. The effect size calculation takes the difference in the mean Indevus Urgency Severity Scale for baseline and week 12 and divides it by the standard deviation of baseline scores. Responsiveness was assessed three times using three external criteria to classify the patients as responders or not.

The first effect size assessment examined the responsiveness of the IUSS by normal toilet void frequency outcome, using the criteria below:
Normal Void Responders with an average of ≦7 toilet voids per 24 hours during week 12.
Non-responders with an average of >7 toilet voids per 24 hours during week 12.

The effect size for the responders was 1.17, which is considered very large, indicating that the IUSS is highly responsive to a reduction in the patient's average toilet void frequency per 24 hours to ≦7 toilet voids. The non-responders had an effect size of 0.21, which is considered small.

The second effect size assessment examined the responsiveness of the IUSS by urge incontinence outcome, using the criteria below:
Urge Incontinence Responders with an average of <1 urge incontinence event per 24 hours during week 12.
Non-responders with an average of ≦1 urge incontinence event per 24 hours during week 12.
   The effect size for the responders was 0.49, which is considered moderate, indicating that the IUSS is moderately responsive to a reduction in patient's average urge incontinence episodes per 24 hours to <1. The non-responders had an effect size of <0.01, indicating that the scale does not indicate change for patients that did not have a decline in the number of urge incontinence episodes to <1.

The third effect size assessment examined the responsiveness of the IUSS by combining the toilet void and urge incontinence outcomes, using the criteria below:
Complete Responders with an average of ≦7 toilet voids and <1 urge incontinence event per 24 hours during week 12.
Non-responders with an average of ≧1 urge incontinence and/or >7 toilet voids per 24 hours during week 12 (i.e. could be either or both criteria).

The effect size for the responders was 1.39, which is considered very large. This indicates that the IUSS is highly responsive to the combined outcomes, i.e. a reduction in the patient's average toilet void frequency per 24 hours to ≦7 toilet voids and a reduction in patient's average urge incontinence episodes per 24 hours to <1. The non-responders had an effect size of 0.20, indicating that the scale does not indicate change for patients that did not have a decline in either or both criteria.

3.4 Test-Retest Reliability

The test-retest reliability of the IUSS was assessed by comparing scores on the scale from baseline day 1 to baseline day 7. This was assessed for 518 patients. The result for test-retest reliability was moderate (0.66 Pearson's correlation coefficient, 0.63 Spearman's rho) when comparing the average IUSS score from baseline day 1 with baseline day 7. The desired level of reliability is usually 0.80.

Following this, as a result of concerns that the day 1 and day 7 data were influenced due to the clinical study protocol (as discussed in section 4.0) additional post-hoc analysis was undertaken to compare day 2 and day 5 baseline data. The result for test-retest reliability between these two time points was 0.80 [0.78 Spearman's rho], which is the desired level of reliability.

3.5 Sensitivity Analysis

Sensitivity analysis was undertaken to ensure that the combination of the trospium chloride and placebo patients into one patient cohort was a valid approach. To assess this the construct validity assessment was repeated separately on the two patient groups and the p-values were compared when this was assessed using Pearson's correlation coefficient. Results indicate the combination of the two treatment groups was a valid approach for all outcomes except for the male version of the IIQ where the p-values notably differed. Results were more likely to be significant for the active treatment group. It was also notable that the construct validity results were more likely to be significant for placebo group for the average number of toilet voids per 24 hours.

Further investigation compared the p-values from the known group analysis for the two treatment groups when assessed using Pearson's correlation coefficient, and these were found to be the same or similar in all instances.

3.6 Content Validity and Respondent Burden

OAB is characterized by the symptoms of urinary frequency, urinary urgency and urge incontinence which are often associated nocturia that appear without a local pathologic or metabolic explanation^{iv}. Although previous studies have frequently focused on urge incontinence^{v}, including the health-related quality of life of patients with urinary incontinence^{vi}, a recent survey conducted in Europe found that frequency and urgency were almost as common as urge incontinence as a reason for seeking medical help^{vii}. Thus the importance of assessing the severity of urgency in a clinical trial for a treatment for OAB is clearly established. However, it was recently reported that ‘currently, no standardized assessment of the subjective symptom of urgency exists, despite the fact that the prime symptom is included in the overall definition of the syndrome^{viii}.
^{iv}Abrams P, Cardozo L, Fall M, et al. The standardization of terminology of lower urinary tract function: Report from the standardization sub-committee of the International Continence Society. Neurourol Urodyn 2002; 21: 167-178.

^{v}Burglo K, Ouslander J. G., Effects of urge urinary incontinence on quality of life in older people. JAGS 1999; 47: 1032-1033.

^{vi}Shumaker S. A., Wyman J. F., Uebersax J. S., et al. Health-related quality of life measures for women with urinary incontinence: The Incontinence Impact Questionnaire and the Urogenital Distress Inventory. Continence Program for Women (CPW) Research Group. Quality of Life Research 1994; 3: 291-306.

^{vii}Milsom I. The prevalence of overactive bladder. Presented at the 14^{th} Congress of the European Association of Gynecology and Obstetrics, September 1999, Grenada, Spain.

^{viii}Staskin D. R., Dmochowski R. R. Future studies of overactive bladder: the need for standardization. Urology 2002; 60 (supplement 5A): 90-93.

To investigate this claim, a review of the leading patient-reported outcome measures for use amongst patients with OAB was undertaken, and the results summarized below:
Urogenital Distress Inventory (UDI)ⁱⁱ: The UDI was developed through patient interviews and discussions, consultation with clinicians, and a literature review. The UDI consists of 19 items which asks a patient whether they are experiencing a specific symptom at present, and the amount of bother they experience from that symptom. The OAB scale of the UDI consists of 3 questions, namely diurnal frequency, nocturnal frequency and urgency. Each item is measured on a five-point Likert.
   ⁱⁱShumaker S. A., Wyman J. F., Vebersax J. S., McClish D, Fanti J. A., for the Continence Program in Women (CPW) Research Group. Health-related quality of life measures for women with urinary incontinence: the Incontinence Impact Questionnaire and the Urogenital Distress Inventory. Quality of Life Research 1994; 3: 291-306.
IIQⁱⁱ: The IIQ was developed alongside the UDI. The IIQ consists of 30 items that assess the impact of urogenital symptoms on four aspects of quality of life: physical functioning, emotional functioning, travel/mobility, and social functioning. Each item is measured on a four-point likert scale. None of the questions are specifically linked to urgency or other OAB symptoms.
   ⁱⁱShumaker S. A., Wyman J. F., Vebersax J. S., McClish D, Fanti J. A., for the Continence Program in Women (CPW) Research Group. Health-related quality of life measures for women with urinary incontinence: the Incontinence Impact Questionnaire and the Urogenital Distress Inventory. Quality of Life Research 1994; 3: 291-306.
Kings Health Questionnaire (KHQ)^{ix}: The KHQ consists of eight multi-item domains: role limitations, physical limitations; social limitations, personal relationships, emotions, sleep, energy and severity (coping) measures. Two single-item domains assess incontinence impact and general health perception. In addition, a multi-item Severity Symptom Scale measures the severity of urinary symptoms (frequency, urgency, urge incontinence, intercourse incontinence, nocturia, nocturnal enuresis, frequent urinary tract infections, bladder pain and difficulty passing urine). The severity of each of the 10 urinary symptoms are measured on a scale ranging from “1” for “a little” to “3” for “a lot”^{ix}.
   ^{ix}Kelleher C. J., Cardozo L. D., Khullar V, Salvatore S. A new questionnaire to assess the quality of life or urinary incontinent women. Br J Gynaecol. 1997; 104: 1374-1379.
Overactive Bladder Symptom and OABq^{x}: The OABq was developed because there were no continent and incontinent OAB-specific subjective patient-reported outcome measures. The instrument was developed through focus groups of males and females, clinician opinion and a literature review. It consists of eight items on a symptom bother scale, and 25 HRQL items.
   ^{x}Coyne K, Revicki D, Hunt T, et al. Psychometric validation of an overactive bladder symptom and health-related quality of life questionnaire: The OAB-1. Quality of Life Research 2002; 11: 563-574.

This review has shown that most existing symptom-orientated instruments include a single question to assess the severity of urgency, providing notable support for the content validity of the IUSS. However, urgency in the context of OAB has been described as consisting of several components^{viii} with the major descriptors being:
(1) Magnitude of discomfort, duration and frequency; and
(2) How well or how long the patient can suppress this.
   ^{viii} Staskin DR. Dmochowski R R. Future studies of overactive bladder: the need for standardization. Urology 2002; 60 (supplement 5A): 90-93.

It is therefore stated that ‘Specific measures of symptom duration, intensity, and type and ability to delay will be required to appropriately assess urinary urgency and provide a better definition of urge incontinence^{viii} (p.91). Within this framework the IUSS can be described as assessing the ‘magnitude (or severity) of discomfort’ of urgency. It can also be used to determine the frequency of urgency episodes. In the context of the clinical study (#IP631-003), the mean number of times patients indicated an urgency with a severity >0 (i.e. scoring 1, 2 or 3 on the IUSS) during the baseline, week 4 and week 12 periods can be assessed, including calculation of change in the frequency of urgency episodes over the period of the 12-week trial. Results indicate that the mean number of urgency episodes at baseline was 11.72 for the placebo group and 11.29 for the trospium chloride group, reducing by an average of 1.08 urgency episodes for the placebo group and 2.30 for the trospium group, and that these results were significant (p<0.01). However the IUSS does not access information on the duration of the urgency, or how well or how long the patient can suppress this.
^{viii} Staskin DR. Dmochowski R R. Future studies of overactive bladder: the need for standardization. Urology 2002; 60 (supplement 5A): 90-93.

Respondent burden is defined as the time, effort, and other demands placed on those to whom the instrument is administered. Because the IUSS is a single item scale with four possible responses, the respondent burden is minimal. However, through a review of the clinical study protocol (#IP631-003) and patient diaries, it is known that patients are required to complete the IUSS for each toilet void and this will notably increase the burden to the respondents.

Discussion

The psychometric assessment of the IUSS was performed retrospectively on data collected during a 12-week clinical trial for trospium chloride. Thus this analysis had the benefit of utilizing data collected within the careful guidelines set out in protocol #IP631-008, ensuring that the data was of the highest standard. In addition, the content validity and respondent burden of the IUSS was assessed through a review of documents relating to the clinical trial #IP631-008 and a literature review.

The results from the assessment of the construct validity of the IUSS were stronger for the week 12 period than for the baseline period. At week 12, the IUSS had a low-to-moderate correlation with the average number of toilet voids per 24 hours and the average number of urge incontinence episodes per 24 hours. The low-to-moderate correlation coefficients indicate that IUSS partially accesses information on the number of toilet voids and urge incontinence episodes. If the correlation coefficients were too high the IUSS might be considered redundant, which supports the results of the content validity assessment which established the need for an urgency-specific measure for OAB. The average volume voided did not correlate with the IUSS for the baseline or week 12 data, an indication that the IUSS does not access this information.

At week 12 IIQ total score and each IIQ subscale score correlated with the IUSS, indicating overlapping construct between the IUSS and the impact of incontinence on physical activity, emotional health, travel-mobility and social relationships. However, a review of published literature on the IIQⁱⁱ raises concerns about the decisions made for the factor structure of this instrument. A recent publication^{xi} presented a revised factor structure for this instrument with five subscales namely mobility, emotional health, physical health, social health and embarrassment. It will be recommended that the construct validity of the IUSS be re-assessed on the basis of this revised factor structure. It is also essential to note that the IIQ has been validated amongst females only. It is therefore essential that the adapted male version of the IIQ is validated before the results of this content assessment for IIQ scores for males and combined (Female and Male) can be considered truly meaningful.
ⁱⁱShumaker S. A., Wyman J. F., Vebersax J. S., McClish D, Fanti J. A., for the Continence Program in Women (CPW) Research Group. Health-related quality of life measures for women with urinary incontinence: the Incontinence Impact Questionnaire and the Urogenital Distress Inventory. Quality of Life Research 1994; 3: 291-306.

^{xi}Vaart van der C. H., de Leeuw J. R. J., Roovers J. P. W. R., Heintz A. P. M. Measuring health-related quality of life in women with urogenital dysfunction: the Urogenital Distress Inventory and Incontinence Impact Questionnaire revisited. Neurology and Urodynamics 2003; 22: 97-104.

The results from the known group validity assessment were good for both the baseline and week 12 data. At both time points, the IUSS demonstrated the important quality of discriminating patients with an average of <10.4 toilet voids and <0.9 urge incontinence episodes per 24 hours. Known groups validity is an essential quality for any patient reported outcome measure and this is a very encouraging indication that the IUSS is capable of this distinction.

It is essential that a patient reported outcome instrument is responsive to change if it is to be used in clinical trials. The results indicate that the IUSS is highly responsive to change in toilet void frequency (average toilet voids per 24 hours ≦7 versus >7). It is also highly responsive to change in toilet void and urge incontinence frequency combined (average toilet voids per 24 hours ≦7 and average urge incontinence episodes <1, versus not having either or both of these outcomes). The IUSS was moderately responsive to change in urge incontinence frequency (average urge incontinence episodes <1 versus ≧1). Importantly the IUSS did not indicate change where change had not taken place for each of these three outcomes. Although responsiveness is a quality of the measurement instrument, the method chosen to define the changed and unchanged groups is a key determinant of responsiveness. It should be noted that using more liberal criteria will yield lower responsiveness indices^{xii}, and that therefore the strict criteria applied during this assessment partially explains the high responsiveness indices.
^{xii}Birbeck G. L., et al. Quality of life measures in epilepsy. How well can they detect change over time? Neurology 2000; 54: 1822-1827.

Test-retest reliability is an index of temporal stability in that it tells how much the individual's normative score might possibly change on retest if a period of time has elapsed between test administrations. The result for test-retest reliability was moderate (0.66 Pearson's correlation coefficient) when comparing the average IUSS score from baseline day 1 with baseline day 7. The desired level of reliability is usually 0.80. Given this, it was essential to consider what was causing the change in average daily score between baseline day 1 and 7. Random error is the most common cause for diminished questionnaire reliability, caused by poorly worded or presented questions leading to inaccurate answers or responses that cannot be interpreted. If this is the cause of the variation, test-retest reliability can be improved by tightening up administration and training procedures, and undertaking a careful pilot of the questionnaire to determine the cause of the error. However, it is also possible that the variation might have something to do with the condition (i.e. urgency associated with OAB) itself. If the condition is itself unstable, then this will be reflected in the IUSS scores, in other words if the instrument does not measure a stable trait, then it would not be expected to have high test-retest reliability. It is also feasible that the day 1 and day 7 data were influenced due to the clinical study protocol. On days 6 and 7, patients were required to measure their volume voided, which they were not required to collect on day 1, and this could potentially influence the data (i.e. the feeling of urgency). There is also some concern that the day 1 data may not be of the usual pattern because this is the first day the patient begins to record their urgency on the UISS. Alternative comparisons (e.g. day 2 with day 5) may therefore result in improved test-retest reliability. Following this additional post-hoc analysis compared day 2 with day 5 at baseline, and found the test-retest result increased to 0.80, and therefore has achieved the desired level of reliability.

An assessment of the content validity of the IUSS established the importance of this scale for OAB because of a lack of standardized assessments for urgency in OAB patients. This validation demonstrated that the IUSS accesses information on the severity -magnitude of discomfort in urgency and the frequency of urgency episodes associated with toilet voids and micturations. The IUSS does not access information on the duration or how well or long the patient can suppress the urge.

Conclusions

The objective of this study was to determine the psychometric qualities of the IUSS. The IUSS is a single-item scale that has been developed as a self-report instrument for use in clinical trials. This study has demonstrated the instrument to be psychometrically sound in terms of construct, content and known groups validity, test-retest reliability, and to be responsive to change.

Synopsis of Results

Title of study:

A multicenter, double-blind, placebo-controlled study of 20 mg, twice-daily trospium chloride for 12 weeks followed by a 9-month, open-label treatment phase in patients with overactive bladder.

Studied period: Approximately 10 months Phase of development: Phase III

Objectives:

The objective of this study was to determine the effects of 20 mg of trospium chloride versus placebo, given twice daily, on overactive bladder associated with predominant urge incontinence over a 12-week treatment period, followed by a 9-month open-label period of trospium chloride available to all patients who participated in the double-blind period. This study report includes data from the double-blind treatment phase of the study. The results from the open-label treatment phase of this study will be provided in a separate study report after the open-label treatment phase has been completed.

Methodology:

Multicenter, parallel, randomized, double-blind, placebo-controlled trial of patients with overactive bladder (OAB) associated with predominant urge incontinence. Patients who were currently receiving OAB drug therapy were to begin treatment after a 3-week washout period that included the 7-day patient urinary diary collection. Patients who were not currently receiving OAB drug therapy (i.e., naive patients) could begin treatment with study medication following their 7-day patient urinary diary collection and continued for a total double-blind treatment duration of 12 weeks.

Patients were randomized on a 1:1 basis to receive either placebo or trospium chloride 20 mg twice daily during the double-blind treatment segment of the study. The randomization schedule for patients was stratified by the mean baseline number of micturitions (i.e., toilet voids) per 24 hours (collected via the patient urinary diary over 7 days); specifically, using the stratified categories of 10 to 15, 16 to 20, and ≧21 mean micturitions per 24 hours. Patients were periodically evaluated during the 12-week treatment segment and were to return on Day 84 for the Week 12 assessments.

Upon completion of the Day 84 (Week 12) visit, if desired, patients could continue into an open-label overactive bladder trial and receive 20 mg trospium chloride twice daily for up to 9 months. The results from the open-label treatment phase will be provided in a separate report after the open-label treatment phase has been completed.

Number of subjects (planned and analyzed):

Planned: Approximately 510 patients-trospium-255 patients; placebo-255 patients.

Randomized: 523 patients-trospium-262 patients; placebo-261 patients.

Diagnosis and main criteria for inclusion: Subjects with overactive bladder associated with predominant urge incontinence.

Test product, dose and mode of administration, batch number:

Trospium chloride

Oral tablet: 20 mg given twice daily

Batch number: A0104896

Duration of treatment:

12-week, double-blind treatment phase followed by an optional 9-month open-label treatment phase.

Reference therapy, dose and mode of administration, batch number:

Placebo

Oral tablet: one matching placebo tablet given twice daily

Batch number: 30701

Criteria for evaluation:

Efficacy:

Patient urinary diary data were collected over 7 days prior to the baseline, Day 8 (Week 1), Day 28 (Week 4), and Day 84 (Week 12) visits except for volume voided, which was collected for 2 full days prior to each study visit. Change from baseline was analyzed for all weeks; the key efficacy analyses focused on the change from baseline to Week 12. Incontinence Impact Questionnaire (IIQ) was used to assess effects of treatment on the patient's quality of life at baseline and Week 12.

Primary efficacy: The co-primary efficacy variables were:

Change in average number of toilet voids per 24 hours.
Change in average number of urge incontinence episodes per 24 hours.
   Secondary efficacy: The key secondary efficacy variables were:
Change in average volume voided per toilet void.
Change in average urgency severity associated with toilet void. Additional secondary efficacy variables were: onset of action during Week 1 for effect on toilet voids per 24 hours; change in average number of diurnal and nocturnal toilet voids; normal void frequency outcome (average of ≦7 toilet voids) per 24 hours; onset of action during Week 1 for effect on urge incontinence episodes per 24 hours; change in average number of diurnal and nocturnal urge incontinence episodes; urge incontinence responder outcome (average of <1 urge incontinence event) per 24 hours; change in average number of total incontinence episodes (urge and stress incontinence episodes) per 24 hours; change in average number of stress incontinence episodes per 24 hours; change in average number of total micturitions (i.e., toilet voids and urge incontinence episodes) per 24 hours; onset of action during Week 1 for effect on total micturitions per 24 hours; change in average number of diurnal and nocturnal total micturitions; and complete responder outcome (average of ≦7 toilet voids and average of <1 urge incontinence episode) per 24 hours. Additional efficacy criteria for evaluation included IIQ score totals, factors, and items, overall and by gender.
   Safety:

Adverse events (AEs), clinical laboratory tests, vital signs, and 12-lead electrocardiograms (ECGs).

Statistical methods:

The Fisher's Exact test was used to analyze adverse events. Other categorical data were analyzed using the Cochran-Mantel-Haenszel (CMH) procedure with center as the stratification variable. For all continuous data analyses, an analysis of variance (ANOVA) model with center and treatment was used.

Homogeneity of variance was assessed using Levene's test and normality was examined using Q-Q plots of the residuals and the Shapiro-Wilk statistic (p:≦0.10 significance level). If the data were not normally distributed, the Median Test or rank transformations of the original data was used.

The p-values presented throughout the report were two-tailed tests of significance. P-values in the text of the study report were rounded to 2 decimal places and used to define significance. Per protocol, p-values ≦0.05 were considered significant; p-values >0.05 and ≦0.10 were considered to represent a trend toward significance. P-values presented in the tables (i.e., both in-text and end-of-text tables) were rounded to 4 decimal places. Based on an agreement made with the Food and Drug Administration (FDA), no correction for co-primary variables was required.

Summary-Conclusions:

Efficacy results:

Primary efficacy: Trospium demonstrated statistically significant (p≦0.05) improvement (i.e., decrease) for the primary efficacy variables of change in average number of toilet voids per 24 hours (at Weeks 1, 4, and 12) and change in average number of urge incontinence episodes per 24 hours (at Weeks 4 and 12) when compared with the placebo group.

Secondary efficacy: Trospium demonstrated statistically significant improvement for the key secondary efficacy variables of change in average volume voided (in mL) and change in average urgency severity at Weeks 1, 4, and 12 when compared with placebo. Specifically, trospium showed a statistically significant increase in average volume voided and a significant decrease in average urgency severity per 24 hours.

Onset of action during Week 1 for effect on toilet voids, urge incontinence episodes, and total micturitions per 24 hours—trospium was superior when compared with placebo with consistently significant effects by Day 7. The results of these analyses show that the onset of effect for trospium is expected to provide some clinical benefit by significantly decreasing the number of toilet voids, decreasing the number of urge incontinence episodes, and decreasing the number of total micturitions per 24 hours within the first week of treatment.

| | Mean change from baseline | | | |
|---|---|---|---|---|
| Change from baseline: | | Placebo | Trospium | |
| Efficacy endpoint/ | Week | N = 256 | N = 253 | P-value |
| Number of toilet voids/ | 12 | −1.29 | −2.37 | <0.0001 |
| 24 hours^{a} | | | | |
| Number of urge | 12 | −1.98 | −2.20 | 0.0118 |
| incontinence episodes/24 hours^{b} | | | | |
| Volume voided (mL) per | 12 | 7.72 | 32.14 | <0.0001 |
| toilet void/24 hours^{a,c} | | | | |
| Urgency severity score | 12 | −0.04 | −0.22 | 0.0001 |
| associated with toilet voids^{a} | | | | |
| Number of nocturnal | 12 | −0.29 | −0.47 | 0.0255 |
| toilet voids/24 hours^{a,d} | | | | |

^{a}Treatment differences assessed by analysis of variance for ITT:LOCF data set.
^{b}Treatment differences assessed by ranked analysis of variance for ITT:LOCF data set.
^{c}Placebo N = 253, Trospium N = 248.
^{d}Placebo N = 253, Trospium N = 253.
ITT = intent to treat,
LOCF = last observation carried forward data set.

Summary-Conclusions (continued):

There was 1 patient who experienced an adverse event leading to death. An 81-year-old male in the trospium group experienced a hemorrhagic stroke on Day 57. Study medication was discontinued on Day 57. The patient died on Day 125 as a consequence of the hemorrhagic stroke. This event was assessed by the investigator as remotely related to study medication and rather attributed the stroke to amyloid angiopathy with possible hypertensive component. Study medication was permanently discontinued due to TEAEs in a total of 38 patients (trospium 23 patients, 8.8%; placebo 15 patients, 5.7%). The most common TEAEs that led to discontinuation of study medication in the trospium group were dry mouth, constipation, abdominal pain NOS, and urinary retention.

Subgroup analyses were done by patient age, gender, and race for the most common TEAEs (i.e., occurred in ≧2.0% of patients in either treatment group and were reported in more trospium patients than placebo patients). Findings from these analyses showed an overall tendency for a higher percentage of trospium patients in age categories of 65 to <75 years and ≧75 years of age to experience a TEAE when compared with trospium patients <65 years of age. There was a tendency in the trospium group for an increased occurrence of dry mouth and constipation as patient age increased. Subgroup analyses by gender showed a tendency in the trospium group for an increased occurrence of headache in the female patients when compared to male patients. In addition, there was a tendency in the trospium group for an increased occurrence of urinary retention in the male patients when compared to female patients.

Clinical laboratory data: The number of patients who met potentially clinically significant (PCS) criteria for the hematology and serum chemistry laboratory data were similar for the trospium and placebo groups. Although there were more patients in the trospium group who met PCS criteria for urinary WBCs and epithelial cells when compared with the placebo group, the differences in the UA findings did not translate into clinically meaningful differences. Vital signs and ECG data: Overall, the number of patients who met PCS criteria for the vital signs and ECG data were similar for the trospium and placebo groups. Although the number of patients who met PCS criteria for high heart rate (>100 and increase ≧15 bpm) were small, there appeared to be more patients in the trospium group when compared with the placebo group. There were no patients in either treatment group who met PCS criteria for high heart rate of >120 and increase <15 bpm.

There were a total of 2 patients, 1 patient in each treatment group, who had QTcF intervals >500 msec that had increased by >60 msec from baseline. For both of these patients, there were no clinical signs or symptoms associated with the increased QTcF interval, there was no action taken with study medication as a result of the QTcF interval increase, and both of the QTcF interval increases had resolved at endpoint.

Summary-Conclusions (continued):

Conclusion:

Trospium given as 20 mg twice daily was shown to be significantly better than placebo for the 2 co-primary efficacy endpoints of decreased average number of toilet voids/24 hours and decreased average number of urge incontinence episodes/24 hours as well as other endpoints including increased average volume voided/24 hours and decreased average urgency severity associated with toilet voids/24 hours. Trospium was safe and generally well tolerated in this study. The data from this study support the conclusion that trospium 20 mg bid is an effective and generally safe treatment option in patients with overactive bladder with predominant urge incontinence.

### EXAMPLE 2

Patients with overactive bladder associated with predominant urge incontinence and complaining of frequent wakefulness throughout the night due to repeated urges to urinate are given 20 mg of trospium chloride 0.5 h before sleeping. The patients experience uninterrupted sleep throughout their normal sleep periods.

### EXAMPLE 3

People complaining of frequent wakefulness throughout the night due to repeated urges to urinate are given 20 mg of trospium chloride 1.0 h before sleeping. The patients experience uninterrupted sleep throughout their normal sleep periods.

Appendix A: Indevus Urgency Severity Scale for Overactive Bladder

Excerpt from instructions in the patient urinary diary: “. . . after checking off each “TOILET VOID”, rate the “DEGREE OF URGENCY” you felt before making it to the toilet.

“DEGREE OF URGENCY” is meant to describe your urge to urinate. Sometimes you may feel a very strong urge to urinate, and at other times, you may feel a milder urge prior to the onset of a “TOILET VOID”. Rate this feeling by circling 0, 1, 2, or 3 defined as:
0: NONE—no urgency
1: MILD—awareness of urgency, but is easily tolerated and you can continue with your usual activity or tasks.
2: MODERATE—enough urgency discomfort that it interferes with or shortens your usual activity or tasks

3: SEVERE—extreme urgency discomfort that abruptly stops all activity or tasks.

| | | |
|---|---|---|
| URINARY DIARY | DAY 7 | / / |
| | | mm dd yyy |

| | | | |
|---|---|---|---|
| ARISING TIME: | : YAM YPM | BEDTIME: | : YAM YPM |

| | | TOILET | | Voided | ACCIDENTS | |
|---|---|---|---|---|---|---|
| | | Circle | | Volume | URGE | STRESS |
| TIME | | VOID Degree of | | (cc) | Leak | Leak |
| | YAM | | | | | |
| : | YPM | Y | 0 1 2 3 | | Y | Y |
| | YAM | | | | | |
| : | YPM | Y | 0 1 2 3 | | Y | Y |
| | YAM | | | | | |
| : | YPM | Y | 0 1 2 3 | | Y | Y |
| | YAM | | | | | |
| : | YPM | Y | 0 1 2 3 | | Y | Y |
| | YAM | | | | | |
| : | YPM | Y | 0 1 2 3 | | Y | Y |
| | YAM | | | | | |
| : | YPM | Y | 0 1 2 3 | | Y | Y |

### DEGREE OF URGENCY

0: NONE—no urgency
1: MILD—awareness of urgency, but easily tolerated
2: MODERATE—enough urgency discomfort that it interferes with usual activity/tasks
3: SEVERE—extreme urgency discomfort that abruptly stops all activity/tasks

### ACCIDENTS

“URGE Leak”=leakage due to very strong need to urinate
“STRESS Leak”=leakage due to coughing, sneezing, standing, laughing, exercise, or other physical activity/movement exerting pressure on the bladder
   Appendix B: IIQ—Original (Female) Version

Some women find that accidental urine loss and/or prolapse may affect their activities, relationships, and feelings. The questions below refer to areas in your life which may have been influenced or changed by your problem. For each question, check the response that best describes how much your activities, relationships, and feelings are being affected by urine leakage and/or prolapse during the past week.

Has urine leakage and/or prolapse affected your:

| A. | Ability to do household chores (cooking, housecleaning, laundry) | | | |
|---|---|---|---|---|
| | □ | □ | □ | □ |
| | Not at all | Slightly | Moderately | Greatly |
| B. | Ability to do usual maintenance or repair work done in home | | | |
| | or yard | | | |
| | □ | □ | □ | □ |
| | Not at all | Slightly | Moderately | Greatly |
| C. | Shopping activities | | | |
| | □ | □ | □ | □ |
| | Not at all | Slightly | Moderately | Greatly |
| D. | Hobbies and pastime activities | | | |
| | □ | □ | □ | □ |
| | Not at all | Slightly | Moderately | Greatly |
| E. | Physical recreational activities such as walking, swimming, or | | | |
| | other exercise | | | |
| | □ | □ | □ | □ |
| | Not at all | Slightly | Moderately | Greatly |
| F. | Entertainment activities such as going to a movie or concert | | | |
| | □ | □ | □ | □ |
| | Not at all | Slightly | Moderately | Greatly |
| G. | Ability to travel by car or bus for distances less than 20 | | | |
| | minutes away from home | | | |
| | □ | □ | □ | □ |
| | Not at all | Slightly | Moderately | Greatly |
| H. | Ability to travel by car or bus for distances greater than 20 | | | |
| | minutes away from home | | | |
| | □ | □ | □ | □ |
| | Not at all | Slightly | Moderately | Greatly |
| I. | Going to places if you are not sure about available restrooms | | | |
| | □ | □ | □ | □ |
| | Not at all | Slightly | Moderately | Greatly |
| J. | Going on vacation | | | |
| | □ | □ | □ | □ |
| | Not at all | Slightly | Moderately | Greatly |
| K. | Church or temple attendance | | | |
| | □ | □ | □ | □ |
| | Not at all | Slightly | Moderately | Greatly |
| L. | Volunteer activities | | | |
| | □ | □ | □ | □ |
| | Not at all | Slightly | Moderately | Greatly |
| M. | Employment (work) outside the home | | | |
| | □ | □ | □ | □ |
| | Not at all | Slightly | Moderately | Greatly |
| N. | Having friends visit you in your home | | | |
| | □ | □ | □ | □ |
| | Not at all | Slightly | Moderately | Greatly |
| O. | Participating in social activities outside your home | | | |
| | □ | □ | □ | □ |
| | Not at all | Slightly | Moderately | Greatly |
| P. | Relationship with friends | | | |
| | □ | □ | □ | □ |
| | Not at all | Slightly | Moderately | Greatly |
| Q. | Relationship with family excluding husband/companion | | | |
| | □ | □ | □ | □ |
| | Not at all | Slightly | Moderately | Greatly |
| R. | Ability to have sexual relations | | | |
| | □ | □ | □ | □ |
| | Not at all | Slightly | Moderately | Greatly |
| S. | Way you dress | | | |
| | □ | □ | □ | □ |
| | Not at all | Slightly | Moderately | Greatly |
| T. | Emotional health | | | |
| | □ | □ | □ | □ |
| | Not at all | Slightly | Moderately | Greatly |
| U. | Physical health | | | |
| | □ | □ | □ | □ |
| | Not at all | Slightly | Moderately | Greatly |
| V. | Sleep | | | |
| | □ | □ | □ | □ |
| | Not at all | Slightly | Moderately | Greatly |
| W. | Does fear of odor restrict your activities? | | | |
| | □ | □ | □ | □ |
| | Not at all | Slightly | Moderately | Greatly |
| X. | Does fear of embarrassment restrict your activities? | | | |
| | □ | □ | □ | □ |
| | Not at all | Slightly | Moderately | Greatly |

In addition, does your problem cause you to experience any of the following feelings?

| Y. | Nervousness or anxiety | | | |
|---|---|---|---|---|
| | □ | □ | □ | □ |
| | Not at all | Slightly | Moderately | Greatly |
| Z. | Fear | | | |
| | □ | □ | □ | □ |
| | Not at all | Slightly | Moderately | Greatly |
| AA. | Frustration | | | |
| | □ | □ | □ | □ |
| | Not at all | Slightly | Moderately | Greatly |
| AB. | Anger | | | |
| | □ | □ | □ | □ |
| | Not at all | Slightly | Moderately | Greatly |
| AC. | Depression | | | |
| | □ | □ | □ | □ |
| | Not at all | Slightly | Moderately | Greatly |
| AD. | Embarrassment | | | |
| | □ | □ | □ | □ |
| | Not at all | Slightly | Moderately | Greatly |

Appendix C: IIQ—Adapted (Male) Version

The question below refer to areas in your life which may have been influenced by any urine leakage you may have. Check the number that best describes how your activities, relationships, and feelings are affected by urine leakage during the past week.

| A. | Ability to do household chores (cooking, cleaning, laundry) | | | |
|---|---|---|---|---|
| | □ | □ | □ | □ |
| | Not at all | Slightly | Moderately | Greatly |
| B. | Ability to do usual maintenance or repair work in the home, | | | |
| | yard | | | |
| | □ | □ | □ | □ |
| | Not at all | Slightly | Moderately | Greatly |
| C. | Shopping activities | | | |
| | □ | □ | □ | □ |
| | Not at all | Slightly | Moderately | Greatly |
| D. | Hobbies and pastime activities | | | |
| | □ | □ | □ | □ |
| | Not at all | Slightly | Moderately | Greatly |
| E. | Physical recreation (walking, swimming, other exercise) | | | |
| | □ | □ | □ | □ |
| | Not at all | Slightly | Moderately | Greatly |
| F. | Entertainment activities (movies, concerts, etc.) | | | |
| | □ | □ | □ | □ |
| | Not at all | Slightly | Moderately | Greatly |
| G. | Ability to travel by car or bus less than 30 minutes from | | | |
| | home | | | |
| | □ | □ | □ | □ |
| | Not at all | Slightly | Moderately | Greatly |
| H. | Ability to travel by car or bus more than 30 minutes from | | | |
| | home | | | |
| | □ | □ | □ | □ |
| | Not at all | Slightly | Moderately | Greatly |
| I. | Going to places if you are not sure about available restrooms | | | |
| | □ | □ | □ | □ |
| | Not at all | Slightly | Moderately | Greatly |
| J. | Going on vacation | | | |
| | □ | □ | □ | □ |
| | Not at all | Slightly | Moderately | Greatly |
| K. | Church or temple attendance | | | |
| | □ | □ | □ | □ |
| | Not at all | Slightly | Moderately | Greatly |
| L. | Volunteer activities | | | |
| | □ | □ | □ | □ |
| | Not at all | Slightly | Moderately | Greatly |
| M. | Employment (work) outside the home | | | |
| | □ | □ | □ | □ |
| | Not at all | Slightly | Moderately | Greatly |
| N. | Having friends visit you in your home | | | |
| | □ | □ | □ | □ |
| | Not at all | Slightly | Moderately | Greatly |
| O. | Participating in social activities outside your home | | | |
| | □ | □ | □ | □ |
| | Not at all | Slightly | Moderately | Greatly |
| P. | Relationships with friends | | | |
| | □ | □ | □ | □ |
| | Not at all | Slightly | Moderately | Greatly |
| Q. | Relationships with family | | | |
| | □ | □ | □ | □ |
| | Not at all | Slightly | Moderately | Greatly |
| R. | Ability to have sexual relations | | | |
| | □ | □ | □ | □ |
| | Not at all | Slightly | Moderately | Greatly |
| S. | The way that you dress | | | |
| | □ | □ | □ | □ |
| | Not at all | Slightly | Moderately | Greatly |
| T. | Your emotional health | | | |
| | □ | □ | □ | □ |
| | Not at all | Slightly | Moderately | Greatly |
| U. | Your physical health | | | |
| | □ | □ | □ | □ |
| | Not at all | Slightly | Moderately | Greatly |
| V. | Your sleep | | | |
| | □ | □ | □ | □ |
| | Not at all | Slightly | Moderately | Greatly |
| W. | Does fear of odor restrict your activities? | | | |
| | □ | □ | □ | □ |
| | Not at all | Slightly | Moderately | Greatly |
| X. | Does fear of embarrassment restrict your activities? | | | |
| | □ | □ | □ | □ |
| | Not at all | Slightly | Moderately | Greatly |
| Y. | Does this problem cause you to feel nervous or anxious? | | | |
| | □ | □ | □ | □ |
| | Not at all | Slightly | Moderately | Greatly |
| Z. | Does this problem cause you to feel fear? | | | |
| | □ | □ | □ | □ |
| | Not at all | Slightly | Moderately | Greatly |
| AA. | Does this problem cause you to feel frustration? | | | |
| | □ | □ | □ | □ |
| | Not at all | Slightly | Moderately | Greatly |
| AB. | Does this problem cause you to feel anger? | | | |
| | □ | □ | □ | □ |
| | Not at all | Slightly | Moderately | Greatly |
| AC. | Does this problem cause you to feel depressed? | | | |
| | □ | □ | □ | □ |
| | Not at all | Slightly | Moderately | Greatly |
| AD. | Does this problem cause you to feel embarrassment? | | | |
| | □ | □ | □ | □ |
| | Not at all | Slightly | Moderately | Greatly |

TABLE 1
Patient Reported Outcome Data

| | Baseline | Week 12 |
|---|---|---|
| Characteristic | (N = 523) | (N = 523) |
| Average Urgency Severity Associated with Toilet Voids^{a} | | |
| n | 520 | 431 |
| Mean (SE) | 1.77 | 1.62 |
| | (0.54) | (0.65) |
| Median | 1.82 | 1.67 |
| Minimum, Maximum | 0.00, | 0.00, |
| | 3.00 | 3.00 |
| Average Number of Toilet Voids per 24 hours | | |
| n | 520 | 431 |
| Mean (SE) | 12.85 | 10.80 |
| | (2.60) | (2.95) |
| Median | 12.29 | 10.43 |
| Minimum, Maximum | 9.29, | 4.29, |
| | 23.14 | 24.86 |
| Average Number of Urge Incontinence Episodes per 24 hours | | |
| n | 520 | 431 |
| Mean (SE) | 4.11 | 1.83 |
| | (3.13) | (2.56) |
| Median | 3.00 | 0.86 |
| Minimum, Maximum | 0.86, | 0.00, |
| | 21.29 | 14.57 |
| Average Volume Voided (in mL) per Toilet Void | | |
| n | 516 | 424 |
| Mean (SE) | 155.27 | 178.32 |
| | (49.29) | (67.16) |
| Median | 154.88 | 172.40 |
| Minimum, Maximum | 20.18 | 29.58, |
| | 286.11 | 404.09 |
| IIQ Total Score (Female)^{b} | | |
| n | 381 | 334 |
| Mean (SE) | 197.13 | 146.59 |
| | (4.36) | (5.37) |
| Median | 199.72 | 136.39 |
| Minimum, Maximum | 3.33, | 0.00, |
| | 388.89 | 400.00 |
| IIQ Total Score (Male)^{b} | | |
| n | 132 | 114 |
| Mean (SE) | 171.58 | 132.56 |
| | (7.29) | (8.24) |
| Median | 178.89 | 123.19 |
| Minimum, Maximum | 0.00, | 0.00, |
| | 377.50 | 379.44 |
| IIQ Total Score (Combined)^{b} | | |
| n | 513 | 448 |
| Mean (SE) | 190.56 | 143.02 |
| | (3.77) | (4.53) |
| Median | 193.98 | 132.22 |
| Minimum, Maximum | 0.00, | 0.00, |
| | 388.89 | 400.00 |
| IIQ Physical Activity Subscale Score (Females)^{b} | | |
| n | 384 | 334 |
| Mean (SE) | 50.19 | 36.80 |
| | (1.23) | (1.45) |
| Median | 50.00 | 33.33 |
| Minimum, Maximum | 0.00, | 0.00, |
| | 100.00 | 100.00 |
| IIQ Physical Activity Subscale Score (Males)^{b} | | |
| n | 132 | 114 |
| Mean (SE) | 42.09 | 31.63 |
| | (1.99) | (2.35) |
| Median | 44.44 | 27.78 |
| Minimum, Maximum | 0.00, | 0.00, |
| | 100.00 | 100.00 |
| IIQ Physical Activity Subscale Score (Combined)^{b} | | |
| n | 516 | 448 |
| Mean (SE) | 48.11 | 35.48 |
| | (1.06) | (1.24) |
| Median | 50.00 | 33.33 |
| Minimum, Maximum | 0.00, | 0.00, |
| | 100.00 | 100.00 |
| IIQ Travel Subscale Score (Female)^{b} | | |
| n | 383 | 334 |
| Mean (SE) | 55.93 | 41.67 |
| | (1.31) | (1.55) |
| Median | 55.56 | 38.89 |
| Minimum, Maximum | 0.00, | 0.00, |
| | 100.00 | 100.00 |
| IIQ Travel Subscale Score (Male)^{b} | | |
| n | 132 | 114 |
| Mean (SE) | 49.33 | 39.72 |
| | (2.16) | (2.45) |
| Median | 50.00 | 38.89 |
| Minimum, Maximum | 0.00, | 0.00, |
| | 100.00 | 100.00 |
| IIQ Travel Subscale Score (Combined)^{b} | | |
| n | 515 | 448 |
| Mean (SE) | 54.24 | 41.17 |
| | (1.13) | (1.31) |
| Median | 55.56 | 38.89 |
| Minimum, Maximum | 0.00, | 0.00, |
| | 100.00 | 100.00 |
| IIQ Social Relationship Subscale Score (Female)^{b} | | |
| n | 384 | 334 |
| Mean (SE) | 40.27 | 30.84 |
| | (1.17) | (1.39) |
| Median | 40.00 | 26.67 |
| Minimum, Maximum | 0.00, | 0.00, |
| | 100.00 | 100.00 |
| IIQ Social Relationships Subscale Score (Male)^{b} | | |
| n | 132 | 114 |
| Mean (SE) | 36.66 | 27.34 |
| | (1.99) | (2.25) |
| Median | 33.33 | 20.00 |
| Minimum, Maximum | 0.00, | 0.00, |
| | 100.00 | 93.33 |
| IIQ Social Relationships Subscale Score (Combined)^{b} | | |
| n | 516 | 448 |
| Mean (SE) | 39.35 | 29.95 |
| | (1.01) | (1.18) |
| Median | 36.67 | 23.33 |
| Minimum, Maximum | 0.00, | 0.00, |
| | 100.00 | 100.00 |
| IIQ Emotional Health Subscale Score (Female)^{b} | | |
| n | 383 | 334 |
| Mean (SE) | 50.72 | 37.29 |
| | (1.23) | (1.45) |
| Median | 50.00 | 33.33 |
| Minimum, Maximum | 0.00, | 0.00, |
| | 100.00 | 100.00 |
| IIQ Emotional Health Subscale Score (Male)^{b} | | |
| n | 132 | 114 |
| Mean (SE) | 43.50 | 33.88 |
| | (2.15) | (2.29) |
| Median | 41.67 | 29.17 |
| Minimum, Maximum | 0.00, | 0.00, |
| | 100.00 | 100.00 |
| IIQ Emotional Health Subscale Score (Combined)^{b} | | |
| n | 515 | 448 |
| Mean (SE) | 48.87 | 36.42 |
| | (1.08) | (1.23) |
| Median | 50.00 | 33.33 |
| Minimum, Maximum | 0.00, | 0.00, |
| | 100.00 | 100.00 |

PROGRAM h:\analysis\indevus\ddr00038.100\saspgm\analy\006t01.SAS (FILE: 006T01.DOC) (11 Mar. 2003 12:26)
N = Number of enrolled patients (i.e. randomized and dispensed study medication).
n = number of enrolled patients with data
IIQ = Incontinence Impact Questionnaireⁱⁱ
^{a}Higher value indicates greater urgency
^{b}Lower value indicates better quality of life.

TABLE 2a
Construct Validity of the IUSS Using Pearson's Correlation

| | Baseline (N = 523) | | | Week 12¹ (N = 523) | | |
|---|---|---|---|---|---|---|
| Criterion Instruments | n | Correlation² | P³ | n | Correlation² | P³ |
| Average Number of Toilet Voids per 24 hours | 520 | 0.08 | 0.08 | 431 | 0.21 | <0.01 |
| Average Number of Urge Incontinence | 520 | 0.34 | <0.01 | 431 | 0.34 | <0.01 |
| Episodes per 24 hours | | | | | | |
| Average Volume Voided (in mL) per toilet | 516 | 0.05 | 0.31 | 424 | 0.01 | 0.90 |
| void | | | | | | |
| IIQ Total Score (Female) | 381 | 0.24 | <0.01 | 318 | 0.30 | <0.01 |
| IIQ Total Score (Male) | 132 | 0.14 | 0.10 | 108 | 0.25 | 0.01 |
| IIQ Total Score (Combined) | 513 | 0.23 | <0.01 | 426 | 0.29 | <0.01 |
| IIQ Physical Activity subscale score | 384 | 0.18 | <0.01 | 318 | 0.25 | <0.01 |
| (Female) | | | | | | |
| IIQ Physical Activity subscale score | 132 | 0.15 | 0.09 | 108 | 0.17 | 0.08 |
| (Male) | | | | | | |
| IIQ Physical Activity subscale | 516 | 0.19 | <0.01 | 426 | 0.23 | <0.01 |
| score (Combined) | | | | | | |
| IIQ Travel subscale score (Female) | 383 | 0.24 | <0.01 | 318 | 0.32 | <0.01 |
| IIQ Travel subscale score (Male) | 132 | 0.12 | 0.18 | 108 | 0.20 | 0.04 |
| IIQ Travel subscale score (Combined) | 515 | 0.22 | <0.01 | 426 | 0.29 | <0.01 |
| IIQ Social Relationships subscale | 384 | 0.22 | <0.01 | 318 | 0.25 | <0.01 |
| score(Female) | | | | | | |
| IIQ Social Relationships subscale | 132 | 0.08 | 0.38 | 108 | 0.23 | 0.02 |
| score(Male) | | | | | | |
| IIQ Social Relationships subscale | 516 | 0.19 | <0.01 | 426 | 0.25 | <0.01 |
| score(Combined) | | | | | | |
| IIQ Emotional Health subscale score | 383 | 0.20 | <0.01 | 318 | 0.29 | <0.01 |
| (Female) | | | | | | |
| IIQ Emotional Health subscale score | 132 | 0.16 | 0.07 | 108 | 0.31 | <0.01 |
| (Male) | | | | | | |
| IIQ Emotional Health subscale score | 515 | 0.20 | <0.01 | 426 | 0.30 | <0.01 |
| (Combined) | | | | | | |

PROGRAM h:\analysis\indevus\ddr00038.100/saspgm/analy/007t02a.SAS (FILE: 007TO2a.doc) (11 MAR. 2003 17:52)
N = Number of enrolled patients (i.e. randomized and dispensed study medication).
n = number of enrolled patients with data
IIQ = Incontinence Impact Questionnaireⁱⁱ
¹Sensitivity analysis validated inclusion of the active treatment group in this patient cohort.
²Reported correlations are based on Pearson's Product Moment correlation between the IUSS and the corresponding criterion instrument.
³P values are calculated based on the asymptomatic distribution of Pearson correlation.

TABLE 2b
Construct Validity of the IUSS Using Spearman's Rho Correlation

| | Baseline (N = 523) | | | week 12¹ (N = 523) | | |
|---|---|---|---|---|---|---|
| Criterion Instruments | n | Correlation² | P³ | n | Correlation² | P³ |
| Average Number of Toilet Voids per 24 hours | 520 | 0.08 | 0.05 | 431 | 0.23 | <0.01 |
| Average Number of Urge Incontinence | 520 | 0.30 | <0.01 | 431 | 0.36 | <0.01 |
| Episodes per 24 hours | | | | | | |
| Average Volume Voided (in ML) per toilet | 516 | 0.05 | 0.24 | 424 | 0.03 | 0.60 |
| void | | | | | | |
| IIQ Total Score (Female) | 381 | 0.24 | <0.01 | 318 | 0.28 | <0.01 |
| IIQ Total Score (Male) | 132 | 0.14 | 0.10 | 108 | 0.27 | <0.01 |
| IIQ Total Score (Combined) | 513 | 0.23 | <0.01 | 426 | 0.28 | <0.01 |
| IIQ Physical Activity subscale score | 384 | 0.19 | <0.01 | 318 | 0.24 | <0.01 |
| (Female) | | | | | | |
| IIQ Physical Activity subscale score | 132 | 0.16 | 0.06 | 108 | 0.23 | 0.02 |
| (Male) | | | | | | |
| IIQ Physical Activity subscale score | 516 | 0.19 | <0.01 | 426 | 0.24 | <0.01 |
| (Combined) | | | | | | |
| IIQ Travel subscale score (Female) | 383 | 0.25 | <0.01 | 318 | 0.32 | <0.01 |
| IIQ Travel subscale score (Male) | 132 | 0.12 | 0.16 | 108 | 0.22 | 0.03 |
| IIQ Travel subscale score (Combined) | 515 | 0.23 | <0.01 | 426 | 0.29 | <0.01 |
| IIQ Social Relationships subscale score | 384 | 0.22 | <0.01 | 318 | 0.24 | <0.01 |
| (Female) | | | | | | |
| IIQ Social Relationships subscale score | 132 | 0.09 | 0.33 | 108 | 0.28 | <0.01 |
| (Male) | | | | | | |
| IIQ Social Relationships subscale score | 516 | 0.19 | <0.01 | 426 | 0.25 | <0.01 |
| (Combined) | | | | | | |
| IIQ Emotional Health subscale score | 383 | 0.18 | <0.01 | 318 | 0.26 | <0.01 |
| (Female) | | | | | | |
| IIQ Emotional Health subscale score | 132 | 0.17 | 0.06 | 108 | 0.33 | <0.01 |
| (Male) | | | | | | |
| IIQ Emotional Health subscale score | 515 | 0.20 | <0.01 | 426 | 0.28 | <0.01 |
| (Combined) | | | | | | |

N = Number of enrolled patients (i.e. randomized and dispensed study medication).
n = number of enrolled patients with data
IIQ = Incontinence Impact Questionnaireⁱⁱ
¹Sensitivity analysis validated inclusion of the active treatment group in this patient cohort.
²Reported correlations are based on Spearman's Rho rank correlation between the IUSS and the corresponding criterion instrument.
³P values are calculated based on the asymptomatic distribution of Spearman correlation.
PROGRAM h:\analysis\indevus\ddr00038.100\saspgm\analy\008t02b.SAS (FILE: 008TO2b.DOC) (11 MAR. 2003 17:52)

TABLE 3
Known Group Validity¹

| | Study Time | Known | | Sum of | p-values³ | |
|---|---|---|---|---|---|---|
| Efficacy Variable | Point | Group² | n | the Ranks | WMW | CMH |
| Average Number of Toilet | Baseline | Group A | 256 | 32896 | <0.01 | 0.71 |
| Voids Per 24 hours | | Group B | 264 | 102564 | | |
| | Week 12 | Group A | 207 | 21528 | <0.01 | |
| | | Group B | 224 | 71568 | | |
| Average Number of Urge | Baseline | Group A | 253 | 32131 | <0.01 | 0.93 |
| Incontinence Episodes | | Group B | 267 | 103329 | | |
| Per 24 hours | Week 12 | Group A | 211 | 22366 | <0.01 | |
| | | Group B | 220 | 70730 | | |

¹Sensitivity analysis validated the inclusion of the active treatment group in this patient cohort.
²Group A < Sample Median and Group B ≧ Sample Median
Reported two-sided p-values for Wilcoxon-Mann-Whitney (WMW) test are based on normal approximation and includes a continuity correction of 0.5 and for Cochran-Mantel-Haenzel (CMH) are based on Chi-square distribution with df = 1
PROGRAM h:\analysis\indevus\ddr00038.100\saspgm\analy\009t03.SAS (FILE: 009T03.DOC) (11 MAR. 2003 19:25)

Having now fully described the invention, it will be understood by one of skill in the art that the invention may be performed within a wide and equivalent range of conditions, parameters and the like, without effecting the spirit or scope of the invention or any embodiment thereof.

### References

ⁱAbrams P, Cardozo L, Fall M, et al. The standardization of terminology of lower urinary tract function: Report from the standardization sub-committee of the International Continence Society. Neurourol Urodyn 2002; 21: 167-178.

ⁱⁱShumaker S. A., Wyman J. F., Vebersax J. S., McClish D, Fanti J. A., for the Continence Program in Women (CPW) Research Group. Health-related quality of life measures for women with urinary incontinence: the Incontinence Impact Questionnaire and the Urogenital Distress Inventory. Quality of Life Research 1994; 3: 291-306.

ⁱⁱⁱCohen J. Statistical power analysis for the behavioral sciences. New York: Academic Press, 1977:8.

^{iv}Abrams P, Cardozo L, Fall M, et al. The standardization of terminology of lower urinary tract function: Report from the standardization subcommittee of the International Continence Society. Neurourol Urodyn 2002; 21: 167-178.

^{v}Burglo K, Ouslander J. G., Effects of urge urinary incontinence on quality of life in older people. JAGS 1999; 47: 1032-1033.

^{vi}Shumaker S. A., Wyman J. F., Uebersax J. S., et al. Health-related quality of life measures for women with urinary incontinence: The Incontinence Impact Questionnaire and the Urogenital Distress Inventory. Continence Program for Women (CPW) Research Group. Quality of Life Research 1994; 3: 291-306.

^{vii}Milsom I. The prevalence of overactive bladder. Presented at the 14^{th} Congress of the European Association of Gynecology and Obstetrics, September 1999, Grenada, Spain.

^{viii}Staskin D. R., Dmochowski R. R. Future studies of overactive bladder: the need for standardization. Urology 2002; 60 (supplement 5A): 90-93.

^{ix}Kelleher C. J., Cardozo L. D., Khullar V, Salvatore S. A new questionnaire to assess the quality of life or urinary incontinent women. Br J Gynaecol. 1997; 104: 1374-1379.

^{x}Coyne K, Revicki D, Hunt T, et al. Psychometric validation of an overactive bladder symptom and health-related quality of life questionnaire: The OAB-1. Quality of Life Research 2002; 11: 563-574.

^{xi}Vaart van der C. H., de Leeuw J. R. J., Roovers J. P. W. R., Heintz A. P. M. Measuring health-related quality of life in women with urogenital dysfunction: the Urogenital Distress Inventory and Incontinence Impact Questionnaire revisited. Neurology and Urodynamics 2003; 22: 97-104.

^{xii}Birbeck G. L., et al. Quality of life measures in epilepsy. How well can they detect change over time? Neurology 2000; 54: 1822-1827.

1. A method of promoting uninterrupted sleep in a person, comprising:
administering trospium chloride to a person at bedtime in an amount sufficient to inhibit awakening throughout the person's intended sleep period.

2. The method of claim 1, wherein the trospium chloride is administered in a solid dosage form.

3. The method of claim 1, wherein the trospium chloride is administered in a liquid dosage form.

4. The method of claim 1, wherein the trospium chloride is administered at a dose between about 1 mg/kg and about 75 mg/kg.

5. The method of claim 1, wherein the trospium chloride is administered at a dose between about 20 mg/kg and about 50 mg/kg.

6. The method of claim 1, wherein the person is a patient with overactive bladder disease.

7. The method of claim 6, wherein the trospium chloride is administered from 0.5 to 1.0 hour before the patient's bedtime.

8. The method of claim 6, wherein the trospium chloride is administered from 1.5 to 2.0 hour before the person's bedtime.

9. A method for treating the discomfort associated with an urge to urinate, in a person suffering from an overactive bladder, comprising: administering to a person with overactive bladder disease an effective amount of trospium chloride, thereby preventing severe discomfort associated with the urge to urinate.

10. A method for measuring the reduction, if any, in the severity of urgency to urinate caused by a test compound that is administered to a population of patients suffering from overactive bladder, comprising:
conducting a double-blind, placebo-controlled study of a drug therapy for patients suffering from overactive bladder, and having the subjects of the study to record the degree of urgency of their toilet voids by rating the degree of urgency they felt just before reaching the toilet, using a single item urgency severity scale having four response options:
0: None—no urgency,
1: Mild—awareness of urgency, but is easily tolerated and you can continue with your usual activity or tasks,
2: Moderate—enough urgency discomfort that it interferes with or shortens your usual activity or tasks; and
3: Severe—extreme urgency discomfort that abruptly stops all activity or tasks; and determining the reduction in the severity of the urge to urinate caused by the test compound in said population.
